(19) United States
(12) Patent Application Publication
(10) Pub. No.: US 2023/0355844 A1
(43) Pub. Date: Nov. 9, 2023

(54) Electrospun Reinforced Suturable Artificial Cornea and Uses Thereof

(71) Applicant: The Schepens Eye Research Institute, Inc., Boston, MA (US)

(72) Inventors: Sina Sharifi, Boston, MA (US); Hannah Sharifi, Boston, MA (US)

(21) Appl. No.: 18/312,848

(22) Filed: May 5, 2023

Related U.S. Application Data
(60) Provisional application No. 63/338,728, filed on May 5, 2022.

(51) Int. Cl. A61L 27/52; A61F 2/14; A61L 27/22

(52) U.S. Cl. CPC A61L 27/52; A61F 2/142; A61L 27/222; A61F 2240/001; A61L 2430/16; D01D 5/003

(57) ABSTRACT

An implant and method of fabricating an implant for corneal replacement is described. According to aspects of the present disclosure, solution electrospinning, hydrogel perfusion, layer-by-layer stacking, and photo-induced crosslinking are used to generate a hydrogel-nanofiber composite with varying fiber diameters and hydrogel concentrations. The integration of nanofibers into the hydrogel synergistically improves the mechanics and suturability of the construct up to 10-fold and 50-fold, respectively, compared to the hydrogel and nanofiber scaffolds alone, approaching those of the corneal tissue.

### CROSS REFERENCE TO RELATED APPLICATIONS

The present application claims priority to U.S. Provisional Patent Application No. 63/338,728, filed May 5, 2022, which is herein incorporated by reference.

### STATEMENT REGARDING FEDERALLY SPONSORED RESEARCH

This invention was made with government support under EY030553 awarded by the National Institutes of Health. The government has certain rights in the invention.

### BACKGROUND

Corneal diseases are one of the significant causes of vision loss, affecting 9.2 million people by recent estimates. Corneal allograft surgery is the primary solution when conditions permanently damage the cornea's structure. Although donor corneas are more accessible for transplantation in high-resource countries, severe scarcity of high-quality donor tissues persists worldwide, with millions in need of transplantation. Moreover, tissue unsuitability, unaffordable cost, allograft rejection, and donor-related infectious complications exacerbate cornea scarcity. Substantial efforts have recently been dedicated to innovating new strategies, ranging from developing techniques to increase the shelf-life of donated corneas and decellularizing xenografts to engineering novel biomaterials to substitute the damaged cornea effectively. Xenografts are analogous to human tissues anatomically and biomechanically. However, due to the presence of antigenic determinants, their transplantation can result in graft rejection. Various natural-based hydrogels derived from collagen gelatin, fibrin, silk, chitosan, and many others have been used to generate corneal substitutes. However, their inferior mechanical properties have significantly impeded their clinical translation. More importantly, the hydrogels' inability to be sutured to host tissues pose a serious challenge for their transplantation.

When stress is exerted to a focal point of the native tissue (e.g., suturing point), the tissue dissipates that stress first at a macromolecular level structure (e.g., collagen helices), and if the focal stress surpasses a certain level, the exerted force is transferred into a larger system (e.g., collagen fibrils) and spread in a larger area. The lack of such hierarchical architecture to efficiently dissipate exerted stress in the hydrogel makes it fragile in suturing points, preventing its transplantation via suturing. Therefore, the applications of hydrogel-based biomaterials developed as corneal substitutes are limited by the necessity of overlying sutures. However, such overlaying sutures can physically stress the implant, delay the bio-integration and healing process, and lead to corneal astigmatism and opacities. Moreover, the existing corneal substitutes can only be used in partial thickness (lamellar) transplantation and not in full-thickness penetrating keratoplasty (PK) due to their inferior biomechanics and inability to withstand sutures' stress.

Recent advances to reinforce the hydrogels include their integration with carbon nanotubes, nanofibres, exfoliated graphene, microfibres, and woven scaffolds. Solution electrospinning, 3D printing, and melt electrospinning were also shown to generate fiber-reinforced constructs with enhanced mechanical properties. Solution-electrospinning affords mats with smaller fibers that mimic native tissues' extracellular matrix structures and mechanical properties; however, there is limited control over the network architecture. On the other hand, melt electrospinning allows tight control over meshes architectures yet results in larger fibers that may not exhibit a reinforcing effect as the relatively thick fibers do not synergically interact with the hydrogel matrix.

### SUMMARY

The present disclosure addresses the aforementioned drawbacks by providing an artificial medical implant and methods of making an artificial implant to generate a hydrogel-nanofiber composite with enhanced mechanical properties and saturability function, which can be used in one non-limiting embodiment as a corneal construct.

According to aspects of the present disclosure, a method of making a medical implant is described. The method comprises: (a) electrospinning a polymer solution to form a polymer fiber mat; (b) diffusing a solution including a crosslinkable hydrogel into the polymer fiber mat to form a hydrogel-infused mat; and (c) irradiating the hydrogel-infused mat to crosslink the hydrogel and form the medical implant. In one embodiment of the method, step (a) comprises electrospinning the polymer solution to form a plurality of polymer mats. In one embodiment of the method, step (b) comprises diffusing a solution including crosslinkable hydrogel into the plurality of polymer fiber mats to form a plurality of hydrogel-infused mats; and further stacking the plurality of polymer fiber mats to form a stack of hydrogel-infused mats. In one embodiment of the method, step (c) comprises irradiating the stack of hydrogel-infused mats to crosslink the hydrogel and form the medical implant. In one embodiment of the method, step (c) comprises molding the hydrogel-infused mat and irradiating the hydrogel-infused mat to crosslink the hydrogel and form the medical implant. In one embodiment of the method, step (c) comprises irradiating the hydrogel-infused mat to crosslink the hydrogel to form a construct, forming an opening in the construct, filling the opening with an additional crosslinkable hydrogel to form a filled construct, and irradiating the additional crosslinkable hydrogel to form a corneal implant.

In one embodiment of the method, the solution includes poly(e-caprolactone) (PCL). In one embodiment of the method, the solution includes one of a polypeptide biopolymer or a polysaccharide biopolymer. In one embodiment of the method, the solution includes one of gelatin and its derivates. In one embodiment of the method, the solution includes gelatin glycidyl methacrylate (G-GMA). In one embodiment of the method, each polymer mat includes fibers of varying diameters, varying orientations of the fibers, or both. In one embodiment of the method, step (c) comprises irradiating the hydrogel-infused mat using a visible light source. In one embodiment of the method, step (c) comprises irradiating the hydrogel-infused mat using a light emitting diode (LED). In one embodiment of the method, step (b) comprises shaving off an excess of the solution from the hydrogel-infused mat diffusing the solution into the polymer fiber mat.

According to aspects of the present disclosure, a medical implant is described. The medical implant comprises: a polymer fiber stack comprising electrospun polymeric fibers; and a crosslinked hydrogel matrix, wherein the polymer fiber stack is embedded within the crosslinked hydrogel matrix. In one embodiment of the medical implant, the polymer fiber stack comprises a stack of a plurality of polymer mats, each polymer mat comprising the electrospun polymeric fibers. In one embodiment of the medical implant, the electrospun polymeric fibers comprise electrospun poly(e-caprolactone) (PCL) polymer fibers. In one embodiment of the medical implant, the electrospun polymeric fibers include fibers of varying diameters, varying orientations, or both. In one embodiment of the medical implant, the hydrogel matrix includes one of a polypeptide biopolymer or a polysaccharide biopolymer. In one embodiment of the medical implant, the hydrogel matrix includes one of gelatin and its derivates. In one embodiment of the medical implant, the hydrogel matrix includes gelatin glycidyl methacrylate (G-GMA).

In one embodiment of the medical implant, the polymer fiber stack includes an opening, and an additional crosslinked hydrogel matrix positioned in the opening. In one embodiment of the medical implant, the additional crosslinked hydrogel matrix includes one of gelatin and its derivates. In one embodiment of the medical implant, the additional crosslinked hydrogel matrix includes gelatin glycidyl methacrylate (G-GMA). In one embodiment of the medical implant, the additional crosslinked hydrogel matrix is transparent. In one embodiment of the medical implant, the crosslinked hydrogel matrix, the opening, and the additional crosslinked hydrogel matrix are each dimensioned such that the implant is a corneal implant.

In one embodiment, the medical implant is an artificial cornea. Advantages of an artificial cornea according to the present disclosure include, without limitation:
customizable (size, thickness, geometry, application, cell count);
mechanical, optical, chemical, and structural properties can be tailor to match to those of native tissue;
biocompatible;
non-immunogenic;
easy to fabricate, preserve, transport, and implant (suturability, handling);
promote regeneration and integrate with native tissue;
cost-effective;
scalable; and
designable to be multifunctional (can be modified with desired functionalities, such as delivering drugs, factors, etc.).

The technology of the present disclosure also can be used to generate other suturable and transplantable constructs, such as blood vessels, hernia mesh, ear drum, skin, tendons, ligaments, and heart valves. The technology of the present disclosure can also be combined with 3D-bioprinting and other fabrication techniques to generate suturable constructs with precise configuration and functionality for specific biomedical needs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of a fabrication process to generate a hydrogel-nanofiber corneal implant according to aspects of the present disclosure.

FIG. 1A shows a schematic of another fabrication process to generate a hydrogel-nanofiber corneal implant according to other aspects of the present disclosure.

FIG. 2A shows the steps of making a hydrogel-nanofiber implant, according to aspects of the present disclosure.

FIG. 2B shows a continuation of steps from FIG. 2A for making a corneal implant, according to aspects of the present disclosure.

FIG. 3A shows scanning electron microscope (SEM) images of generated from PCL solution in a binary mixture of DMF/CHCl₃ with the 4:0, 3:1, 2:2, 1:3, and 0:4 ratios denoted by DMF, D3C1, D2C2, D1C3, and CHCl₃ in low and high resolutions along with their fiber diameter profiles.

FIG. 3B shows the cross-section SEM of the electrospun PCL mats generated from PCL solution in binary mixture of DMF/CHCl₃ with the 4:0, 3:1, 2:2, 1:3, and 0:4 ratios denoted by DMF, D3C1, D2C2, D1C3, and CHCl₃.

FIG. 3C shows the fiber diameter profiles of the PCL solution in a binary mixture of DMF/CHCl₃ with the 4:0, 3:1, 2:2, 1:3, and 0:4 ratios of FIG. 3A

FIG. 3D shows the polydispersity indexes of the nanofibers of FIG. 3A.

FIG. 3E shows the ultimate tensile strength of the electrospun PCL mats generated from PCL solution in binary mixtures DMF/CHCl₃ with varying ratios as in FIG. 3A. 25% (w/w) G-GMA hydrogel solution is denoted as G25. Synergy group=PCL/G25 hybrid construct−(PCL+G25) values.

FIG. 3F shows the tensile modulus of the electrospun PCL mats generated from PCL solution in binary mixtures DMF/CHCl₃ with varying ratios as in FIG. 3A. 25% (w/w) G-GMA hydrogel solution is denoted as G25. Synergy group=PCL/G25 hybrid construct−(PCL+G25) values.

FIG. 3G shows the compressive modulus of the electrospun PCL mats generated from PCL solution in binary mixtures DMF/CHCl₃ with varying ratios as in FIG. 3A. 25% (w/w) G-GMA hydrogel solution is denoted as G25. Synergy group=PCL/G25 hybrid construct−(PCL+G25) values.

FIG. 3H shows the contact angle of the electrospun PCL mats generated from PCL solutions as in FIG. 3A.

FIG. 3I shows the BSA permeability of the electrospun PCL mats generated from PCL solutions as in FIG. 3A.

FIG. 4A shows an experimental set-up for measuring the tensile modulus.

FIG. 4B shows representative tensile stress/strain curves of the constructs (where G10, G15, G20, and G25 stand for G-GMA with 10%, 15%, 20%, and 25% (w/w) concentrations) compared to G25, electrospun PCL, and native human cornea (HC).

FIG. 4C shows the corresponding mean ultimate tensile strength of the constructs of FIG. 4B.

FIG. 4D shows the corresponding tensile modulus (D) of the constructs of FIG. 4B.

FIG. 4E shows the experimental set-up for measuring the constructs' compressive modulus.

FIG. 4F shows the corresponding compressive modulus values of the experimental set-up of FIG. 4E.

FIG. 4G shows SEM micrographs showing the interlayer space.

FIG. 4H shows the corresponding interlayer space values of the SEM micrographs of FIG. 4G.

FIG. 4I shows the experimental set-up for measuring the adhesion between layers of PCL/hydrogel for the constructs made in varying applied forces.

FIG. 4J shows the adhesion strength of PCL/hydrogel constructs of shaved-off hydrogel from perfused PCL mats.

FIG. 4K shows the adhesion strength of PCL/hydrogel constructs of varying hydrogel concentrations (10-25%) of perfused PCL mats.

FIG. 4L shows the experimental set-up for measuring the burst pressure of the constructs (PCL/G25) with varying trephination size (mm).

FIG. 4M shows the burst pressure of the experimental set up of FIG. 4L.

FIG. 4N shows the experimental set-up for measuring the suture cutting force of the constructs (O) compared to those of G25 and HC.

FIG. 4O shows the suture rupture force of the experimental set-up of FIG. 4N.

FIG. 5A shows swelling ratios of the constructs made with varying concentrations of hydrogel (10-25%) in PBS at 37° C. as a function of incubation time.

FIG. 5B shows the retention in the solution containing collagen against biodegradation of the constructs made with varying concentrations of hydrogel (10-25%) in PBS at 37° C. as a function of incubation time.

FIG. 5C shows the glucose diffusion of the constructs made with varying concentrations of hydrogel (10-25%) in PBS at 37° C. as a function of incubation time.

FIG. 6A shows representative live-dead images of human cornea stromal cells (HCS), their corresponding viabilities, and confluencies cultured on the constructs (G25 and PCL/G25) after 1, 4, and 7 days of cell culture.

FIG. 6B shows representative live-dead images of human cornea epithelial cells (HCEp), their corresponding viabilities, and confluencies cultured on the constructs after 1, 4, and 7 days of cell culture (green (calcein-AM): lived cells; red (ethidiumhomodimer-1): dead cells.

FIG. 6C shows the quantification of the metabolic activity of HCS cultured on specimens after 1, 4, and 7 days of cell culture, using AlamarBlue assay.

FIG. 6D shows the quantification of the metabolic activity of HCEp cultured on specimens after 1, 4, and 7 days of cell culture, using AlamarBlue assay.

FIG. 7 shows representative fluorescent immune-stained images of HCS cells cultured on the constructs (G25, G25/PCL, tissue culture plate: TCP, and PCL groups) after 6 days of incubation in the culture media. HCS cells cultured on G25, G25/PCL highly expressed ALDH3A1 (red) and Integrin β 1 (red), FAK (red), Ki67 (red) but not α-SMA in comparative level to those on TCP. HCS cells cultured on PCL weakly expressed ALDH3A1 (red) and Integrin β 1 (red), FAK (red), Ki67 (red), and α-SMA (red). All cell nuclei were counterstained using DAPI (blue). The scale bar is 100 μm.

FIG. 8 shows Representative TEM images of the cross-sectional interface of tissue-construct (G25/PCL) after 2-month incubation in culture media (ex vivo model). The red arrows show PCL nanofibers that some of them were surrounded by the newly formed collagen; white arrows indicate the degradation of G-GMA hydrogel, which is noted by pink arrows; green arrows show the formation of newly synthesized partially organized collagen fibers; and yellow arrows show highly organized collagen fibers, indicating step-by-step hydrogel degradation and tissue regeneration.

FIG. 9A shows porosity of the PCL electrospun nanofibers generated in different binary solvents of DMF, D3C1, D2C2, D1C3, and CHCl₃.

FIG. 9B shows pore size (B) of the PCL electrospun nanofibers generated in different binary solvents of DMF, D3C1, D2C2, D1C3, and CHCl₃.

FIG. 10 shows Stress/Strain plots for PCL nanofibers made in binary solvents of DMF, D3C1, D2C2, D1C3 and CHCl₃.

FIG. 11 shows Stress/Strain plots for PCL/hydrogel composite with varying nanofiber diameters made in different binary solvents of DMF, D3C1, D2C2, D1C3, and CHCl₃.

FIG. 12A shows the UV-Vis spectrum of BSA-FITC conjugate. and its

FIG. 12B shows the calibration plot of the UV-Vis spectrum of BSA-FITC conjugate.

FIG. 12C shows BSA-FITC conjugate diffusion as a function of time for varying reinforced composites.

FIG. 12D shows glucose diffusion as a function of time for varying reinforced composites.

FIG. 13 shows G-GMA to PCL ratios of the composites made in varying hydrogel concentrations in the dry and hydrated state.

FIG. 14A shows the distribution of the fibers inside the construct with varying interlayer space along the Z axis. The force to remove the hydrogel solution from the soaked PCL mats is 0.5 N.

FIG. 14B shows the distribution of the fibers inside the construct with varying interlayer space along the Z axis. The force to remove the hydrogel solution from the soaked PCL mats is 2N.

FIG. 14C shows the distribution of the fibers inside the construct with varying interlayer space along the Z axis. The force to remove the hydrogel solution from the soaked PCL mats is 5 N.

FIG. 15 shows an experimental set-up and corresponding compressive modulus of the constructs (G10, G15, G20, and G25 stand for G-GMA with 10%, 15%, 20%, and 25% (w/w) concentrations) compared to G25, electrospun PCL, and native human cornea (HC) at 25° C. and 37° C. A Thermal-Lok dry heat bath temperature was adjusted to 37° C. and used as a stationary base. The specimens were submerged in PBS, placed on an Aluminum block, and allowed to reach 37° C., followed by compression by a mechanical tester.

FIG. 16 shows example embodiments of hydrogel-nanofiber blood vessels according to aspects of the present disclosure.

### DETAILED DESCRIPTION

The formulation uses solution electrospinning, hydrogel perfusion, and layer-by-layer stacking to generate a hydrogel-nanofiber composite with enhanced mechanical properties and suturability function, which can be used as a corneal construct.

In a non-limiting example, poly(e-caprolactone) (PCL) is used for electrospinning to function as the strong component, and gelatin glycidyl methacrylate (G-GMA) hydrogel-is used as a biodegradable matrix. Alternatively, other useful hydrogels include hydrogels having gelatin and acrylate groups, and hydrogels having gelatin and methacrylate groups. Further, the hydrogel solution may include other polypeptide or polysaccharide biopolymers, such as, but not limited to, collagen, and alginate.

In a non-limiting example, electrospun mats with varying fiber diameters are fabricated by solution electrospinning of medical-grade PCL dissolved in DMF/CHCl₃ solutions with differing ratios. Hydrogel composites are then fabricated by infusing the PCL mats with G-GMA solution, followed by layer-by-layer stacking, compression, and photo-induced crosslinking.

In a non-limiting example, human corneal stromal cells (HCS) and human corneal epithelial cells (HCEp) are cultured on those constructs for the corneal implants. Alternatively, fibroblasts, endothelial cells, or other cell type may be cultured on the construct based on the target implant location and anatomy.

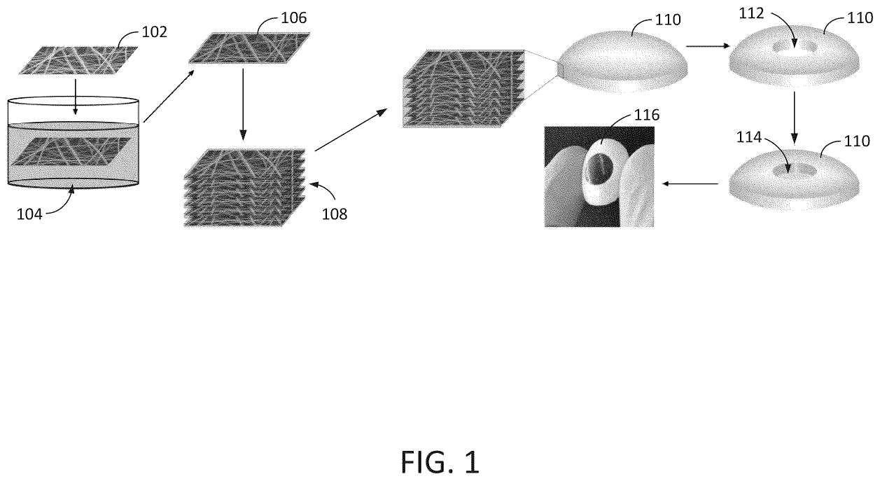

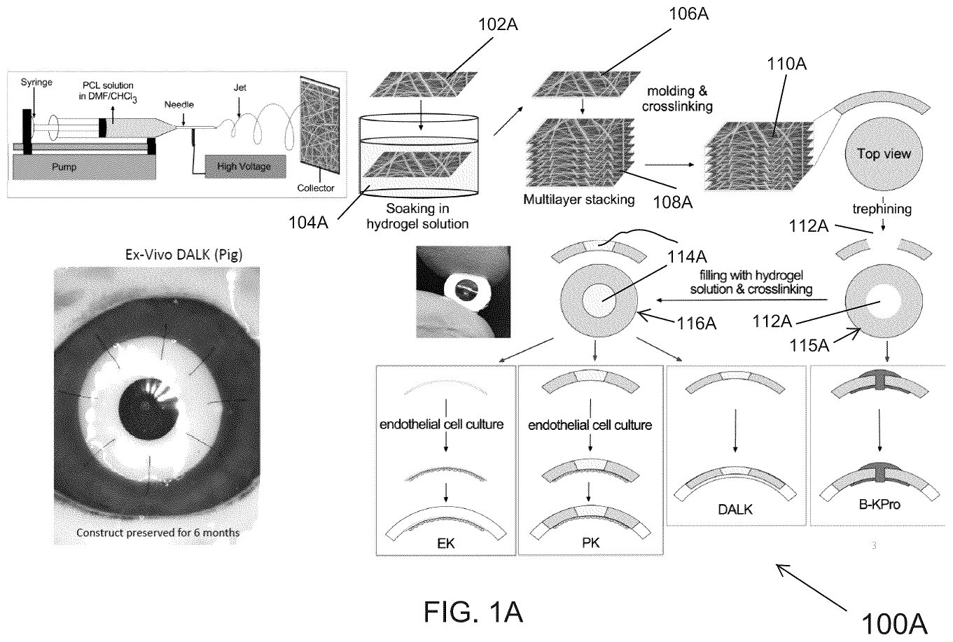

In a non-limiting example, FIG. 1 shows a workflow 100 for fabricating the hydrogel-nanofiber corneal implant. Electrospun mats 102 with varying fiber diameters (e.g., 1 nm. to 10 μm, or 0.1 μm to 20 μm) are fabricated by solution electrospinning of medical-grade PCL dissolved in DMF/CHCl₃ solutions with differing ratios. Hydrogel composites are then fabricated by infusing the PCL mats with G-GMA solution 104 to form hydrogel-infused mats 106. The hydrogel-infused mats 106 are stacked layer-by-layer stacking to form a stack 108, compressed, and irradiated for photo-induced crosslinking. This results in a construct 110. In a non-limiting embodiment for producing a corneal insert, the resulting construct 110 is trephined to form an opening 112, filled with the hydrogel solution 114, and further crosslinked to produce the artificial corneal implant 116. The hydrogel solution 114 for filling the construct may be the same as the hydrogel solution 104 infused within each mat 106. Alternatively, the hydrogel for filling the construct 114 may differ in the type of biopolymer or solution percentage from the hydrogel infused within each mat 104.

In another non-limiting example, FIG. 1A shows a workflow 100A for fabricating another hydrogel-nanofiber corneal implant. Electrospun mats 102A with varying fiber diameters (e.g., 1 nm. to 10 μm, or 0.1 μm to 20 μm) are fabricated by solution electrospinning of medical-grade PCL dissolved in DMF/CHCl₃ solutions with differing ratios. Hydrogel composites are then fabricated by infusing the PCL mats with G-GMA solution 104A to form hydrogel-infused mats 106A. The hydrogel-infused mats 106A are stacked layer-by-layer stacking to form a stack 108A, compressed, and irradiated for photo-induced crosslinking. This results in a construct 110A. In a non-limiting embodiment for producing a corneal insert, the resulting construct 110A is trephined to form an opening 112A, filled with the hydrogel solution 114A, and further crosslinked to produce the artificial corneal implant 116A. The hydrogel solution 114A for filling the construct may be the same as the hydrogel solution 104A infused within each mat 106A. Alternatively, the hydrogel for filling the construct 110A may differ in the type of polymer (e.g., polyvinyl alcohol) or solution percentage from the hydrogel infused within each mat 104A. The artificial corneal implant 116A may be used in endothelial keratoplasty (EK), or penetrating keratoplasty (PK), or deep anterior lamellar keratoplasty (DALK). Alternatively, the trephined construct 115A may be used in Boston keratoprosthesis (B-KPro) implantation.

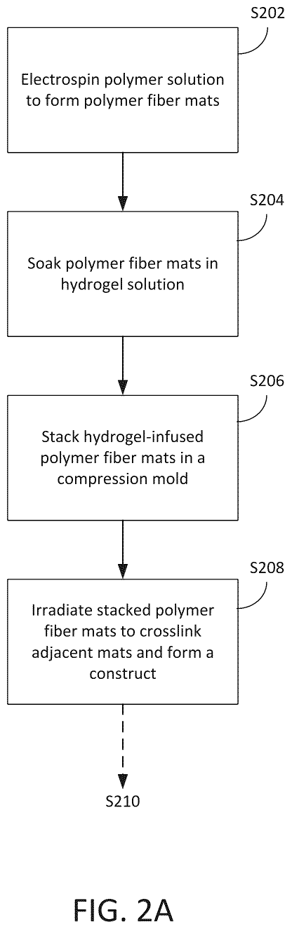

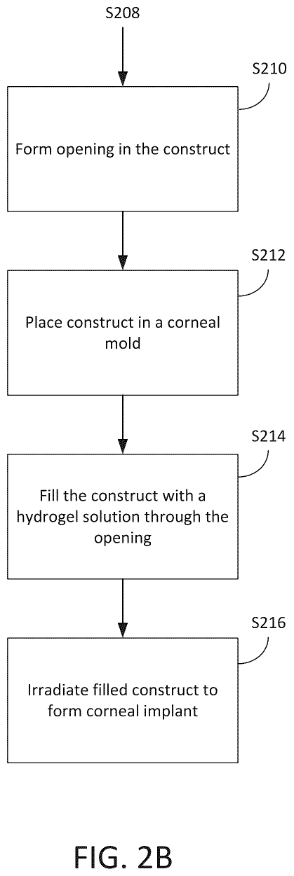

A non-limiting method for making a corneal implant (FIG. 1) is shown in FIGS. 2A-2B. Steps S202-S208 of FIG. 2A may be followed to fabricate a variety of implants such as, but not limited to, tendons, ligaments, skin, heart valves, and blood vessels. At step S202, a polymer solution is electrospun to form one or more polymer fiber mats. The polymer fiber mats are then soaked in a hydrogel solution at step S204. At step S206, the hydrogel-infused polymer fiber mats are stacked and placed in a compression mold followed by irradiation with visible light to crosslink the adjacent mats in the stack. This results in a single hydrogel-nanofiber construct.

FIG. 2B further shows the steps of forming a corneal implant as described in FIG. 1. At step S210, the construct from S208 is trephined to form an opening. The construct is then placed in a corneal mold, which has the dimensions of a human cornea. The corneal mold may be of a discrete size range based on the age and size of a patient. Alternatively, the corneal mold may be custom made for a specific patient. At step S214, the construct is filled through the opening with a hydrogel solution. At step S216, the filled construct is irradiated with visible light for further crosslinking. The resulting corneal implant includes a skirt structure mimicking the mechanical properties of a natural cornea, and a core structure comprising the hydrogel filling for optical light transmission.

In a non-limiting example, the PCL mat has an ultimate tensile strength in a range of 2.5 to 5.5 MPa. In one embodiment, the ultimate tensile strength is in a range of 3 to 5 MPa.

In a non-limiting example, the PCL mat has a tensile modulus in a range of 2.5 to 5.5 MPA. In one embodiment, the tensile modulus is in a range of 4 to 5 MPa.

In a non-limiting example, the PCL mat has a compressive modulus in a range of 2 to 4 MPa. In one embodiment, the compressive modulus is in a range of 2.5 to 3.5 MPa.

In a non-limiting example, the PCL mat has a contact angle in a range of 130° to 135°. In one embodiment, the contact angle is in a range of 131° to 133°.

In a non-limiting example, the PCL mat has a BSA permeability in a range of 5 to 20 cm²/s. In one embodiment, the BSA permeability is in a range of 10 to 15 cm²/s.

In a non-limiting example, adhesion strength between the polymer fiber mat and the crosslinked hydrogel matrix is in a range of 0.2 to 0.7 MPa. In one embodiment, the adhesion strength is in a range of 0.3 and 0.6 MPa.

In a non-limiting example, the implant has a burst pressure in a range of 75 to 275 kPa with varying trephination diameter size ranging from 4 to 10 mm. In one embodiment, the burst pressure is in a range of 100 to 260 kPa.

In a non-limiting example, the implant has a suture rupture force in a range of 3 to 6N. In one embodiment, the suture rupture force in in a range of 4 to 5 N.

In a non-limiting example, the implant has a glucose diffusion in a range of 2 to 4 cm²/s. In one embodiment, the glucose diffusion is in a range of 2.5 to 3.5 cm²/s.

Further details of the corneal implant and the methods of making the corneal implant are further described in the following Example.

### EXAMPLE

The following Example is provided in order to demonstrate and further illustrate certain embodiments and aspects of the present invention and is not to be construed as limiting the scope of the invention. The statements provided in the example are presented without being bound by theory.

### Methods

### Chemical Synthesis G-GMA and Crosslinking Conditions

Gelatin (10 g, 300 g Bloom, type A) was dissolved in 100 mL PBS solution, followed by adding 10 mL of glycidyl methacrylate. The resultant mixture was agitated for 5 h at 45° C., followed by adding 100 mL distilled water and dialysis for 7 days (molecular weight cut-off of 14 kDa; Sigma-Aldrich). The solution was then lyophilized for 4 days to obtain a foam-like G-GMA. To prepare the G-GMA crosslinking solutions, different amounts of G-GMA (1.0, 1.5, 2.0, and 2.50 g) was dissolved in varying amount of PBS (8.36, 7.86, 7.36, 6.86 mL, respectively) at 45° C., followed by the addition of 0.50 g of VP, 100 μL of TEOA, and 40 μL of Eosin Y solution (1 mM)) in dark conditions and agitation at 45° C.

### Electrospinning Conditions and Construct Generation

To prepare electrospinning solutions, 20 g of polycaprolactone (PCL; Mn=80,000) was dissolved in DMF (80 g), a mixture of DMF/CHCl₃ (40/40 g), or CHCl₃ (80 g) overnight at 50° C. in a sealed container to create 20% (w/w) solutions. The solutions were charged in a 10 mL syringe, loaded in a syringe pump, and pumped at varying rates (1-10 μL/min). The electrospinning process was performed at varying voltage (7-20 kV), with the 19 g needle and nozzle to collector distance of 5-20 cm. After collecting the PCL sheets, they were submerged in G-GMA solutions and gently mixed, allowing the hydrogel to diffuse into the PCL mats. Next, the extra G-GMA was gently shaved off the PCL mats, stacked in an appropriate mold compressed, and exposed to visible light (20 mW/cm² LED) for 5 min. To generate an artificial cornea after crosslinking process, the central part of the construct was trephined. Then, the trephined construct was transferred to a corneal mold, and the trephination area was filled with G-GMA solution (25% w/w) and exposed to visible light (20 mW/cm² LED) for another 5 minutes crosslinking to yield a core/skirt structure, in which the skirt can be used for suturing, and the core can transmit the light to provide vision.

### Mechanical Characterization

Tensile and compression tests were conducted on a mechanical tester (Mark-10 ESM 303; Copiague, NY), equipped with MESURgauge Plus software. For the tensile test, after preparing dumbbell-shaped samples (PCL, G-GMA, hybrid construct), they were secured to the mechanical tester grips and extended at a 5 mm/min rate until rupture. The stress was recorded as a function of the strain. The elastic moduli were calculated from the linear derivatives of the stress-strain curves at 25-70% of strain [n=4].

For the compression test, after preparing disc-shaped samples, they were placed on the stationary stage and compressed at the rate of 0.5 mm/min until the maximum stress of 3 MPa. 5 The moduli were computed from the linear derivatives of the stress-strain plots at 0-20% strain [n=4].

For the suture test, after preparing rectangular-shaped samples, they were secured to the mechanical tester mobile grip, followed by passing 6-0 prolene suture through the construct and extension at a rate of 5 mm/min until rupture [n=4].

For the lab shear adhesion test, PCL mats were first cut to rectangular shapes, bathed in hydrogel solutions, cleaned with plastic blade to shave off extra hydrogel, and stacked in a single-lap configuration (˜25 mm2 shear area), followed by gentle compression and exposure to visible light for 5 min. Then, specimens were secured to the mechanical tester grips and stretched at a 5 mm/min rate until separation. All hydrogel-based samples were incubated in PBS at 37° C. for 2 h before running the mechanical tests.

For the burst pressure, first, the artificial corneal constructs were made as described above. After incubation in PBS at 37° C. for 2 h, they were secured in the artificial chamber. The syringe was set to pump PBS (0.2 mL/min) into the chamber, and the burst pressure was measured with a pressure sensor (PS-2017, PASCO; Roseville, CA) and recorded by computer via the PASCO Capstone interface.

### Swelling Ratio

After preparing disc-shaped samples, they were washed with PBS, blot-dried, and weighed to find their initial weights (Wi). Next, they were submerged in PBS and incubated at 37° C. for up to 48 h, blot-dried, and their swollen weights (Ws) were measured. The swelling ratios (S) of the samples [n=4] were assessed using the following equation:

$$S ( \% ) = \frac{( {W}_{s} - {W}_{i} )}{{W}_{i}} \times 100 .$$

### In Vitro Biodegradation

Disc-shaped samples were generated, washed with PBS, and incubated at 37° C. in collagenase from Clostridium histolyticum solution (5 U/mL) in Tris-HC1 buffer (0.1M, pH=7.4), supplemented with CaCl₂ (5 mM), with collagenase solution renewing every 8 h. At each time point, the solution was removed, the residues were lyophilized, and their dried weight (Wf) was measured. The initial weight of samples was assessed using the dried weight (Wi) of untreated specimens. The retention was calculated [n=4] using the following equation:

$$Retention ( \% ) = \frac{{W}_{f}}{{W}_{i}} \times 100 .$$

### Diffusivity Measurements

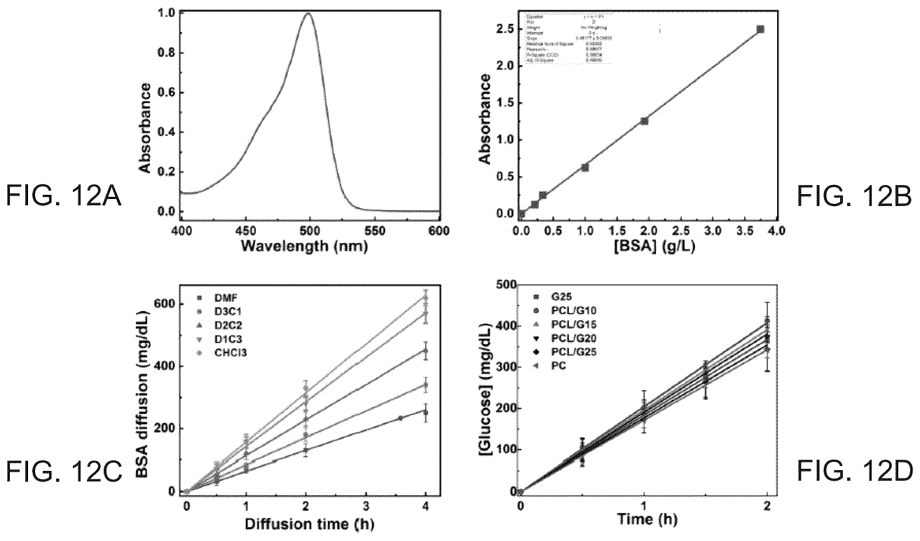

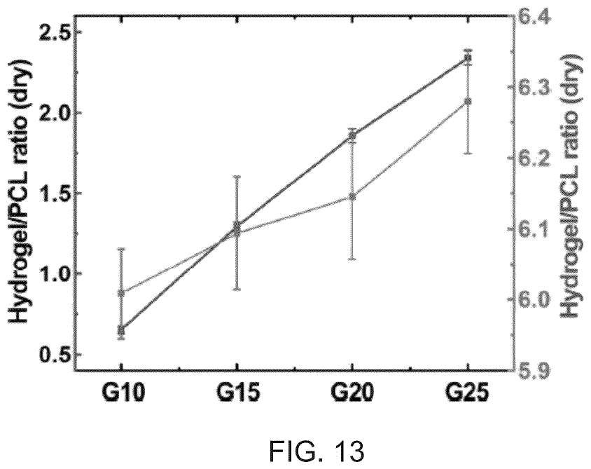

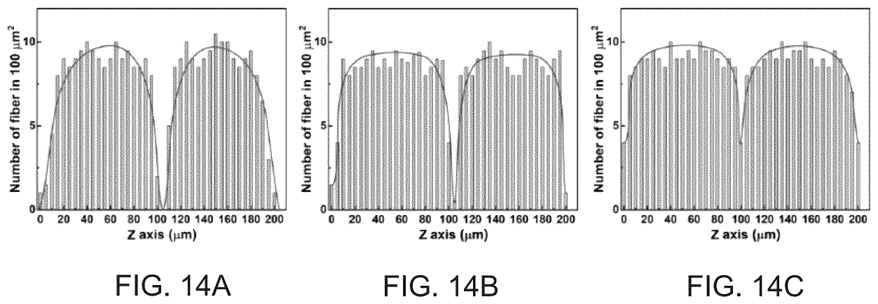

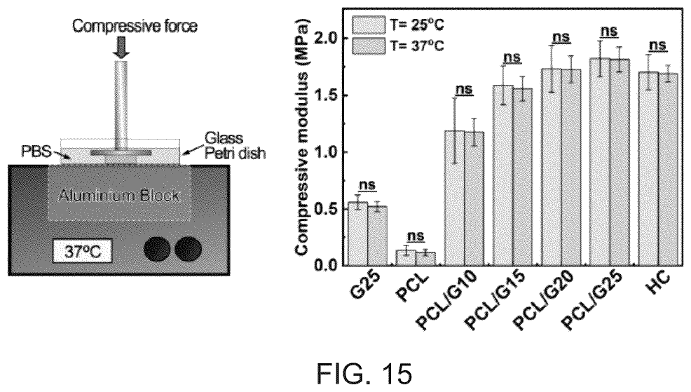

To evaluate the permeability of the samples, a Static Franz cell with a diameter of 9 mm (PermeGear, PA, USA) is used. After preparing disk-shaped samples, they were inserted between the two compartments of the Franz cell. The upper unit was filled with 1 mL PBS and the bottom filled with either [glucose]=2000 mg/d or [BSA-FITC conjugate]=2000 mg/dL. The unit was placed inside an incubator at 37° C., and solutions in both units were stirred using a magnetic stirrer. The [glucose] in the upper unit was determined using a Counter Next EZ blood glucometer (Bayer, Parsippany, NJ, USA) at different time points. The [BSA-FITC conjugate] was assessed by measuring the absorption at 499 nm (λₘₐₓ) using a UV-Vis spectrometer (Molecular Devices SpectraMax 384 Plus Microplate Reader, Molecular Devices; San Jose, CA) and calculating it via a calibration plot (see FIG. 12). The diffusion coefficients were calculated for the samples as described below.

### Calculation of The Diffusion Coefficients

To calculate the diffusion coefficients (D) for each specimen, we used the following mathematical formula, derived from Fick's law of diffusion according to a previously reported method.

Q=DC₁×t/L

where the Q is the amount of glucose (BSA) that passes through the construct during the time (t) per unit area, C₁ is the glucose (BSA) concentration in the lower chamber (2000 mg/dl), L is the thickness of the construct and t is time. From the glucose (BSA) concentration measurements, linear curves for the upper chamber glucose (BSA) concentration versus time were graphed. For each sample, the average glucose (BSA) concentration in the upper chamber at a given time point (0-4 h) and corresponding standard deviations were calculated. The slopes of these lines were then used to obtain the change in concentration per time, which was then divided by the cross-sectional area of the blind-well chamber available for diffusion (0.63 cm²) to obtain Q. Using the above equation, Q, and the average thicknesses of the constructs, the average D values and corresponding standard deviations were calculated for each type of samples.

### Scanning Electron Microscopy (SEM)

Hybrid constructs were frozen in the dry ice, snapped to expose their cross-sections, and lyophilized. Then dried samples or electrospun PCL specimens coated with Au using a sputter coater and imaged using a field emission scanning electron microscope (JEOL NeoScope JCM-7000 SEM; Peabody, MA). Fiber size was extracted and quantified using ImageJ software (NIH, Bethesda, Maryland) from multiple images acquired from each sample (n=4).

### Water Contact Angle Measurement

The water contact angle measurements were performed using a custom-made contact angle goniometer and a static sessile drop technique. At room temperature, a 5 μL size droplet of distilled water was delivered by a syringe, located above the sample surface, onto specimens. A high-resolution camera (Dino-Lite Edge, AM73915MZTL 5MP, Torrance, CA) was then used to capture the image from the side. The contact angle for each group was calculated and averaged from the images acquired from multiple samples (n=4) using ImageJ software via the contact angle plugin.

### In Vitro Biocompatibility

Live-Dead Assay. To assess the cytotoxicity of the specimens and their interactions with human corneal epithelial cells (HCEp), we conducted a standard Live-Dead assay. After preparing disc-shaped specimens, they were submerged in an antibiotic solution comprising 300 unit/mL penicillin and 300 μg/mL streptomycin solution. Then, those discs were washed and used as substrates for culturing (5,000 cells), followed by the addition of appropriate media, 54 (400 μL), and incubation at 37° C. in 5% CO2. After 1, 4, and 7 days of culture, the cells on the specimens were stained with a standard Live-Dead staining kit (LIVE/DEAD™ viability/cytotoxicity kit, Thermofisher Scientific; Cambridge, MA) and imaged by inverted fluorescent microscope (Zeiss Axio Observer Z1; Thornwood, NY). Viabilities were calculated using ImageJ software from multiple images obtained from each specimen (n=4) and compared to those cultured on TCP as a control.

Alamarblue Assay. To evaluate cultured cells' metabolic activity (HCEp and HCS), we used a standard AlamarBlue assay. After culturing cells (HCEp and HCS; 5,000 per well), the AlamarBlue assay was conducted on days 1, 4, and 7 of post culture. At each point, the culture media was replaced with fresh media (400 μL) containing resazurin sodium salt (0.004% w/v) and incubated for 3 h. Afterward, 100 μL of the media was transferred to a 96 well plate, and the fluorescence intensities were measured on a BioTek plate reader (Synergy 2, BioTek Instruments; Winooski, VT) with excitation of 530/25 nm and emission of 600/25 nm and corrected with the fluorescence of corresponding constructs without cells incubated in AlamarBlue assay media (n=6).

Immunocytochemistry (ICC): The expression of specific markers (ALDH3A1, integrin b1, FAK, Ki67, and α-SMA) by HCS cultured on constructs was evaluated by fluorescence ICC. Briefly, after culturing the HCS on the discs for 6 days, they were removed from the media, gently rinsed with PBS, and fixed in 4% paraformaldehyde. After permeabilization with 0.1% Triton X-100, the unspecific protein binding was blocked using 1% Bovine serum albumin (BSA) in PBS. Then, the specimens were incubated with the following primary antibodies for 2 h at 37° C.: (i) mouse monoclonal antibody against ALDH3A1 (clone 1B6; GTX84889, dilution 1:100, GenTex); (ii) rabbit polyclonal antibody against Integrin β1 (GTX112971, dilution 1:250, GenTex); (iii) rabbit monoclonal antibody against FAK (clone EP695Y; ab40794, dilution 1:250, Abcam); (iv) rabbit polyclonal antibody against Ki67 (ab15580, dilution 1:1000, Abcam); or (v) mouse monoclonal antibody against α-SMA (clone 1A4; ab7817, dilution 1:200, Abcam). Then, the discs were incubated with corresponding secondary antibodies: Alexa Fluor 633 anti-mouse IgG2a antibody (A21136, dilution 1:500, Life Technologies); Alexa Fluor Plus 594 anti-rabbit IgG antibody (A32740, dilution 1:500, Life Technologies) for 1 h at 37° C. Finally, the specimens were mounted in VectaShield mounting media containing 4′,6-diamidino-2-phenylindole (DAPI, Vector Laboratories) and imaged using a Leica TCS SP8 confocal microscope (Buffalo Grove, IL).

### Transmission Electron Microscopy (TEM)

After culturing the implanted construct for 2 months, specimens were PBS washed and fixed with Karnovsky's fixative (50% strength at pH=7.4) (Electron Microscopy Sciences, Hatfield, PA) overnight at room temperature. They were then washed with 0.1 M Cacodylate Buffer (Electron Microscopy Sciences) for 5 min, followed by PBS wash three times. The samples were post-fixed with 2% osmium tetroxide for 1.5 h at room temperature, then en bloc stained with 2% aqueous uranyl acetate for 30 min, dehydrated in ethanol, and embedded in epoxy resin (Tousimis, Rockville, MD). Ultrathin sections (80 nm) were cut from each sample using a Leica EM UC7 ultramicrotome (Leica Microsystems, Buffalo Grove, IL). The sections were then stained with gadolinium (III) acetate hydrate (2.5%) and Sato's lead citrate using a modified Hiraoka grid staining system56 and imaged on Hitachi HT7800 TEM (Tarrytown, NY) at 80 kV.

### RESULTS and Discussion

### Fabrication of PCL Nanofibers and Optimization

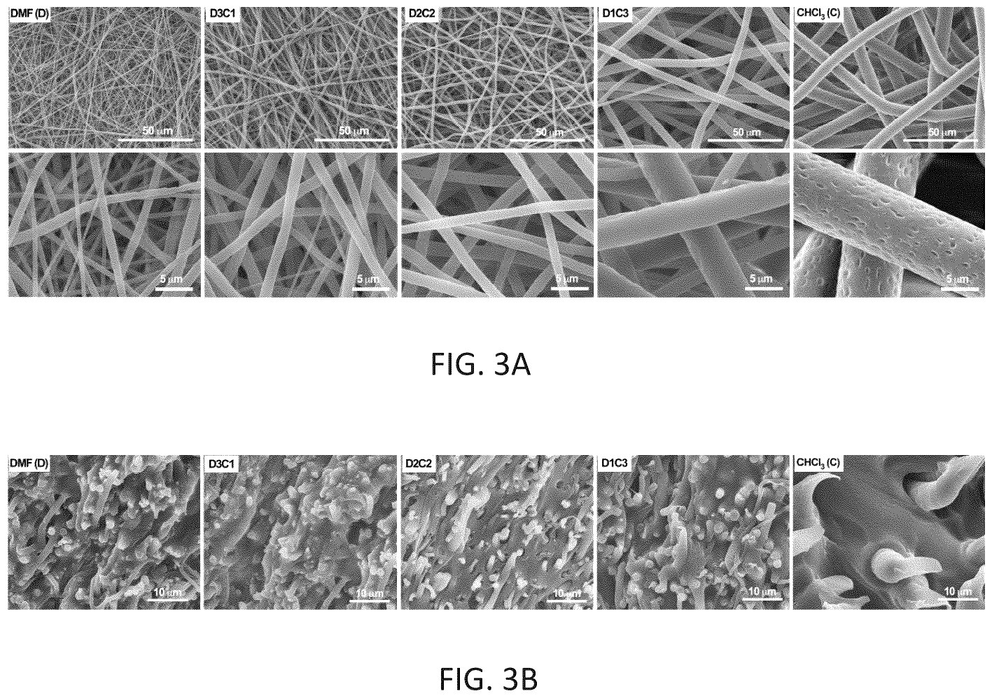

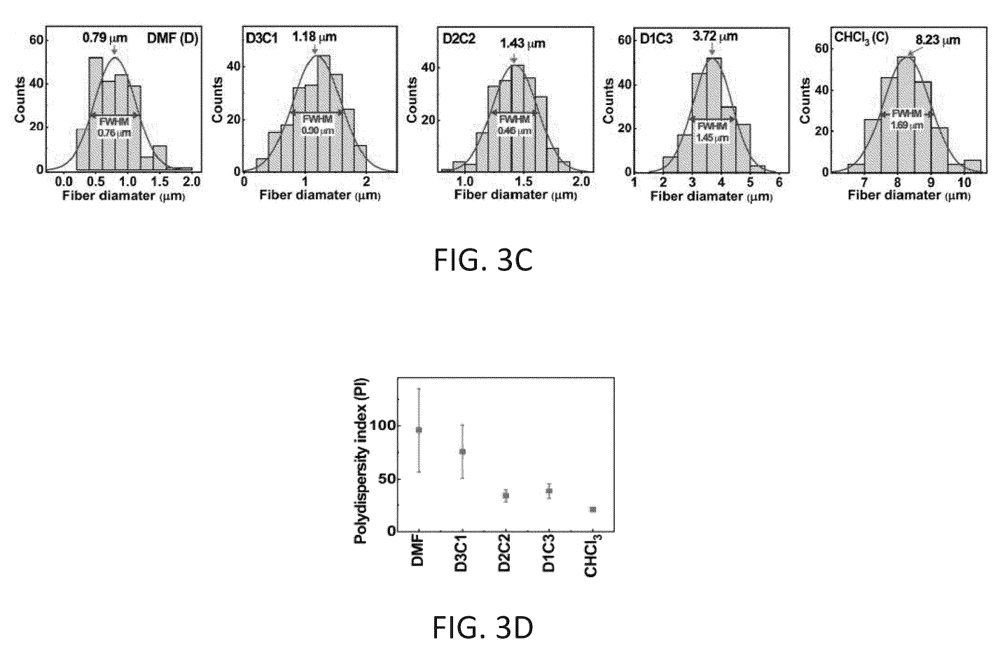

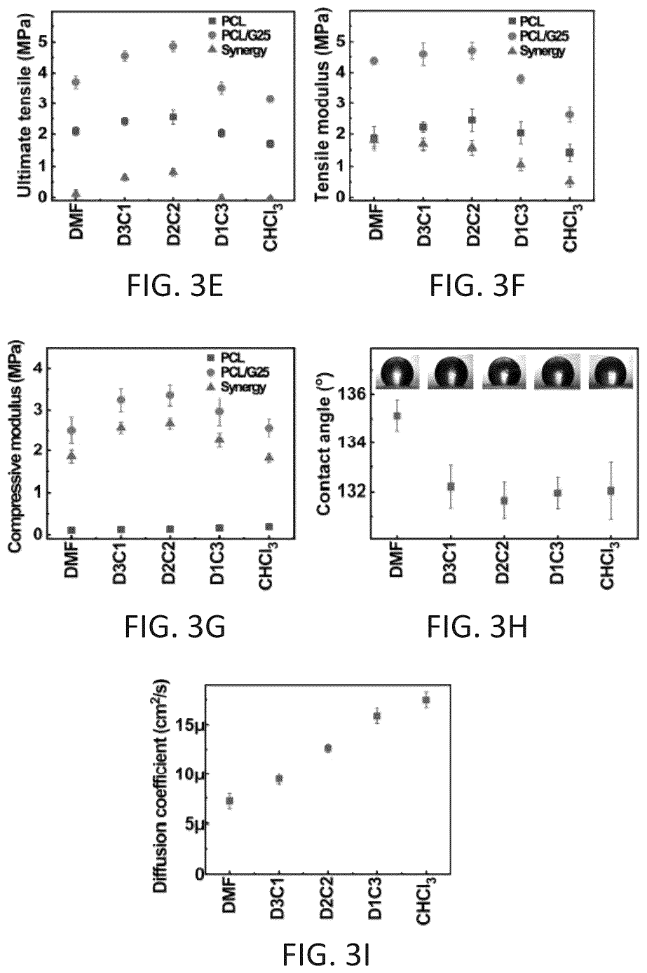

Many parameters govern the electrospinning process, including solution properties, applied voltage, the solution flow rate, collector distance, and nozzle to collector distance. After comprehensive tuning and optimization of all the parameters, a 13 kV applied voltage was used between nozzle and collector that was covered by a single layer of polyethylene plastic (100 μm thickness) to deposit fibers, with a 13 cm distance between nozzle and collector, and a flow rate of 5 μL/min. It was shown that the fiber diameter impacts the properties of the electrospun nanofibers. To obtain the PCL nanofibers with different diameters, we used DMF/CHCl₃ solutions with varying ratios (DMF:CHCl₃=4:0 (DMF(D)), 3:1 (D3C1), 2:2 (D2C2), 1:3 (D1C3), and 0:4 (CHCl₃ (C)), keeping the PCL concentration constant at 17% w/w. SEM analysis shows that the PCL dissolved in the binary system of DMF:CHCl₃ (i.e., 4:0, 3:1, 2:2, 1:3, 0:4 ratios) resulted in nanofibers with a diameter of 0.79±0.32 μm, 1.18±0.39 μm, 1.42±0.22 μm, 3.72±0.56 μm, and 8.23±0.70 μm, respectively. This data suggests that increasing the CHCl₃ ratio led to fibers with larger diameters with a lower polydispersity (FIGS. 3A and 3C-3D). After electrospinning and fabricating PCL mats with a thickness of ca. 100 μm, they were perfused with 25% G-GMA solution, containing the Eosin Y crosslinking reagent, stacked, compressed, and crosslinked to generate ca. 1 mm thickness composite construct.

### PCL Mats Characterization

SEM micrographs show that the G-GMA hydrogel is perfused into PCL mats to form bicomponent composites with varying fiber sizes (FIG. 3B).

PCL mat produced from PCL dissolved in binary mixtures of DMF:CHCl₃ exhibit higher tensile strength and modulus compared to those produced from PCL dissolved in a DMF or CHCl₃ (FIGS. 3E-3F). For instance, mats made from PCL dissolved in a DMF:CHCl₃ with 1:1 ratio (D2C2) showed the highest tensile strength (2.55±0.24 MPa) and modulus (2.44±0.35 MPa). And mats made from PCL dissolved in a DMF or CHCl₃ showed lowest tensile strength (1.68±0.13 MPa) and modulus (1.42±0.25 MPa). Compressive modulus also showed similar trends, with the highest modulus for the D2C2 group and the lowest in DMF (FIG. 3G).

Nanofibers produced from DMF solution have inhomogeneous diameters along the fibers and have higher polydispersity (PD) (FIG. 3D). The addition of CHCl₃ improves the homogeneity of the fibers and lowers the polydispersity in a CHCl₃ content-dependent manner. These inhomogeneities and higher PD can lead to a decline in the mechanical properties of the mats produced in the solvents with higher DMF content. On the other hand, nanofibers produced in the systems with higher concentrations of DMF show some degree of fiber connectivity. Such fiber interconnectivity can enhance the mechanical properties of the mats produced in solvents with higher DMF contents. The higher elongation at break in the D1C3 and CHCl₃ groups can be attributed to the paucity or lack of fiber connectivity. The balance between these two counteracting factors (i.e., fiber connectivity and homogeneity) results in mats with the optimal mechanical properties, as demonstrated in the D2C2 group.

### PCL/Hydrogel Reinforced Composites Characterization (Varying Fiber Diameter)

PCL mats infused with G-GMA (25% w/w) solution exhibited substantially better mechanical properties compared to PCL mats (FIG. 3E-3G), with the highest ultimate tensile of 4.87±0.17 MPa, the tensile modulus of 4.70±0.28, and the compressive modulus of 3.35±0.26 for D2C2 composite group. The data analysis suggests that, in addition to the additive effect, the perfusion of mats with the hydrogel leads to synergistic improvement in the composite mechanics in a nanofibers' structure-dependent manner; the D2C2 composite group showed the greatest synergistic improvement in both ultimate tensile and compressive modulus. Mats with thicker fibers (e.g., CHCl₃ group) exhibited less synergistic improvement, highly likely due to the weaker fiber-hydrogel interactions originating from the lower surface area of the fibers and lack of fiber interconnection and subsequently their entanglement with the hydrogels. Mats with thinner fibers might not have complete perfusion due to the smaller pore size of the mats.

Contact analysis (FIG. 3H) shows that mats with smaller fiber sizes generally have higher water contact angles than those with thicker fibers.

Bovine serum albumin (BSA) diffusion studies (FIG. 3I) demonstrate the following trend: D_{DMF}=7.21·10⁻⁶±0.26·10⁻⁶ cm²/s<D_{D3C1}=9.48·10⁻⁶±0.20·10⁻⁶ cm²/s<D_{D2C2}=1.26·10⁻⁵±0.02·10⁻⁵ cm2/s<D_{D1C3}=1.58·10⁻⁵±0.03·10⁻⁵ cm²/s<D_{CHCl3}=1.74·10⁻⁵±0.03·10⁻⁵ cm²/s (FIG. 3H). This trend is consistent with their apparent pore size distribution.

### PCL/Hydrogel Reinforced Composites Characterization (Varying G-GMA Concentrations)

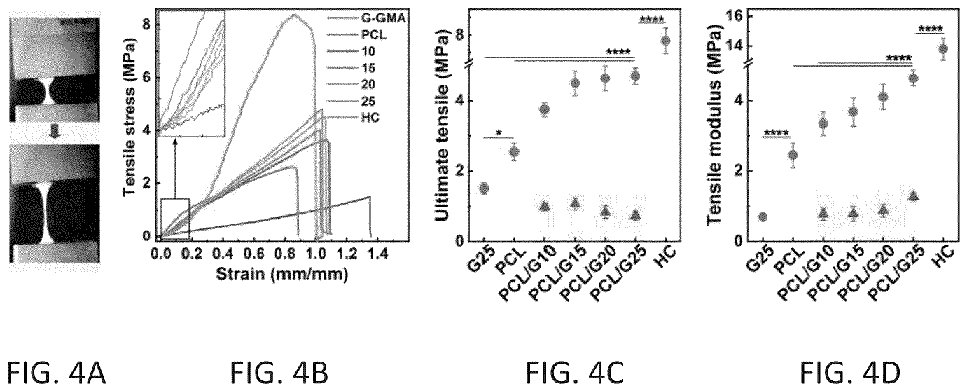

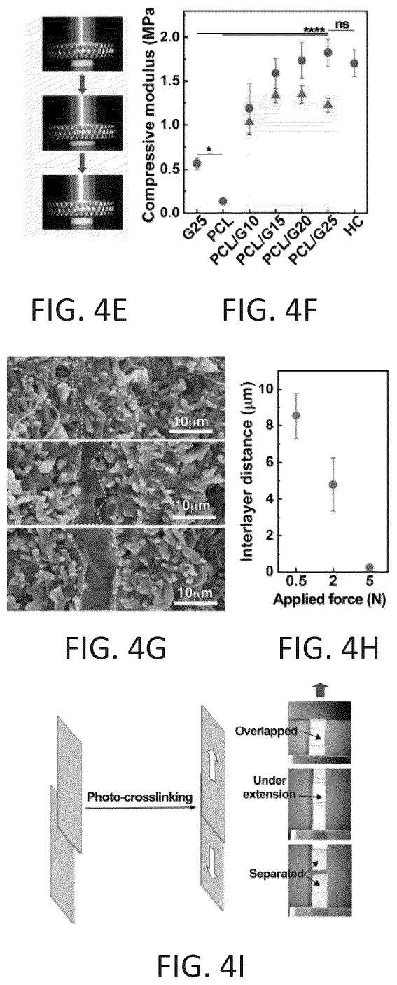

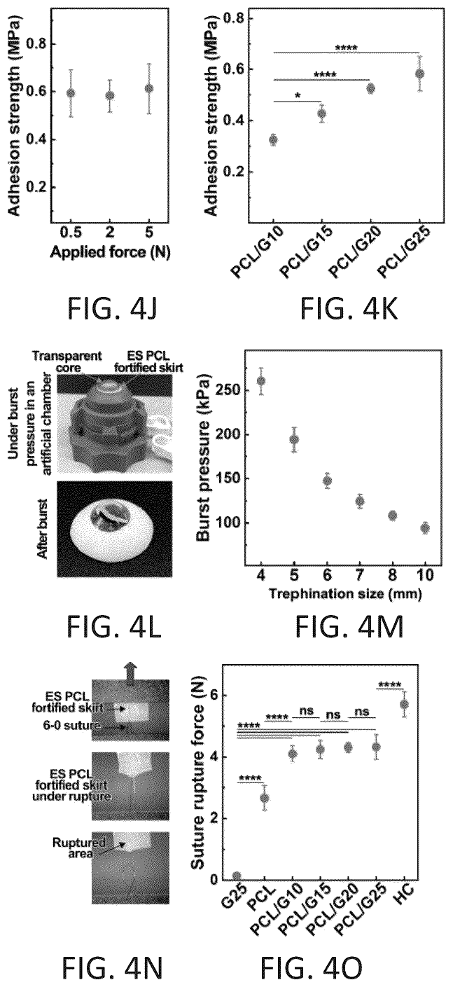

Tensile and compressive properties of the G-GMA composites with varying G-GMA concentrations (i.e., [G-GMA]=10%: G10, [G-GMA]=15%: G15, [G-GMA]=20%: G20, [GGMA]=25%: G25) are illustrated in FIGS. 4A-O. Electrospun reinforced constructs demonstrated significantly better mechanical properties than PCL or G-GMA in a [G-GMA]-dependent manner. The highest strength (4.63±0.35 MPa) was observed for the composite PCL/G25 compared to G25 (1.5±0.15 MPa), PCL (2.53±0.24 MPa), and human cornea (HC) (7.67±0.75 MPa) (FIGS. 4A-4D). A strong synergistic effect was observed on the composite's ultimate tensile in all concentrations of G-GMA (ca. 1 MPa), as highlighted in FIG. 4C. Tensile modulus exhibited a similar ascending trend as a function of [hydrogel], with the highest modulus (4.62±0.22 MPa) for PCL/G25 compared to G25 (0.70±0.05 MPa), PCL (2.44±0.36 MPa), and human cornea (HC) (13.78±0.72 MPa). Tensile modulus also exhibited a strong synergistic effect in all concentrations of G-GMA, ranging from 0.77 MPa (PCL/G10) to 1.28 MPa (PCL/G25), as highlighted in FIG. 4D. In the toe region, the composite's tensile stress/strain behavior ranged between PCL (strong component) and hydrogel (weak component)—the stress/strain behavior of the composite with lower [hydrogel] is closer to PCL, and that with higher [hydrogel] is closer to hydrogel a shown in FIG. 4B inset. Electrospun reinforced constructs also exhibited significantly higher compressive modulus than that of G25 (0.56±0.06 MPa) or PCL (0.13±0.04 MPa), with a strong synergistic effect (FIGS. 4E-4F). PCL electrospun fibers were also infused with homogenized cartilage-derived matrix and exhibited a compressive modulus of ca. 0.04 MPa. Moreover, PCL electrospun mats were infused with poly(ethylene glycol)-diacrylate with varying fiber percentages (0-50%) and crosslinked to fabricate composite gels for cartilage tissue engineering with the compressive modulus of (0.05-0.16 MPa). PCL electrospun mats were also infused with collagen/fibrin to afford hybrid scaffolds for cartilage tissue engineering applications with tensile modulus and strength of ca. 1.76 MPa and 1.11 MPa, respectively. After perfusion of the PCL mats, the remaining hydrogel solutions (G10, G15, G20, and G25) on the PCL mats were removed through a plastic blade, followed by stacking (10 layers in this study) and photo-induced crosslinking. The remaining hydrogel solution on the surface of the layers can act as an adhesive and covalently bond the hydrogel perfused layers to form a unified structure upon light exposure. The thickness of the adhesion layer can be controlled by the amount of pressure applied to the blade to clean off the extra hydrogel, as shown in FIGS. 4G-4H. Having some space between PCL layers allows cells to migrate between layers, degrade the hydrogel and generate ECM to enable biointegration.

To understand the adhesion strength between layers of the construct, we used an adapted lap shear test (FIG. 4I). Our data showed the thickness of the adhesion layer does not impact the adhesion between layers, yet there is a direct correlation between [hydrogel] and the interlayer adhesion strength (FIGS. 4J-4K).

Normal intraocular pressure (IOP) of the eye is 1.3-2.8 kPa and, therefore, the construct used in penetrating keratoplasty should be able to tolerate normal IOP and its fluctuations. In addition, the application of excessive force/pressure in an asymmetric fashion due to eye rubbing—can elicit IOP elevations of ˜10.0-20 kPa above baseline for 3-4 s, with peak IOP elevations reaching 27-41 mmHg—trauma, or compression, could rupture the implant at the suturing points. The burst pressure test showed that the engineered core-skirt architecture—the skirt is made of electrospun reinforced G25, and the core is made of G25—can tolerate extremely high pressures, ranging from 94 kPa to 260 kPa for 4-10 mm trephinations in a trephination size-dependent fashion (FIGS. 4L-4M). These pressures are far above the eye's normal intraocular pressure (IOP) and its fluctuations. Interestingly, in none of the tested samples, the burst occurred at the intersection of skirt-core, indicating the strong core attachment to the skirt through covalent bonding. Suturability testing showed that electrospun reinforced composites have significantly (up to 30-fold) higher resistance to scaffold tearing (˜4.32 N for PCL/G25 group) than G25 hydrogel (˜0.15 N) and approaches that of HC (˜7.45 N) for as shown in FIGS. 4N-4O. Such improvement against suture-induced pressure rupture is attributed to the presence of robust PCL nanofibers organization that can distribute the applied stress of sutures in a larger area and make the construct robust and notch insensitive.

### Structural Properties of Reinforced Composites

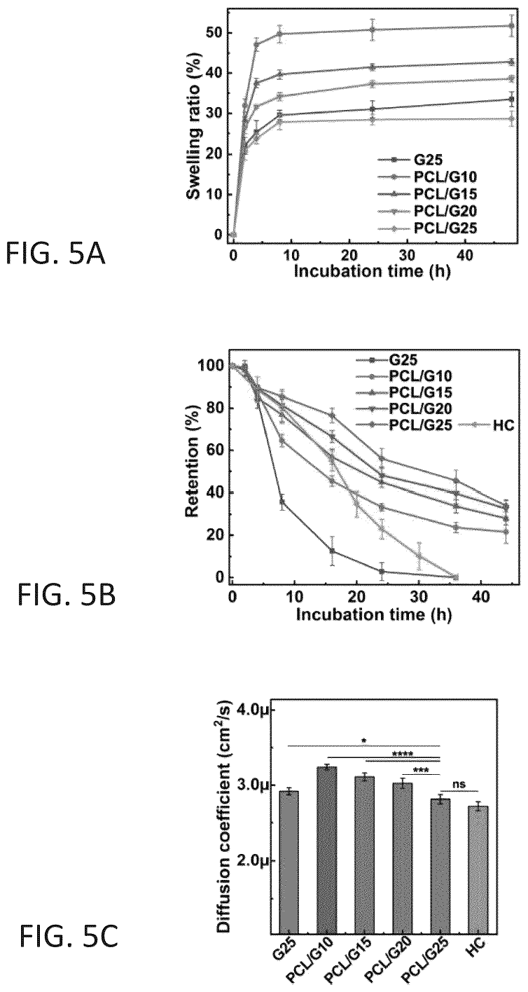

The tissue (e.g., cornea) substitute should have a relatively low swelling ratio to prevent the construct protrusion from its implanted location or its deformation upon swelling, leading to astigmatism and myopia. FIG. 5A shows swelling ratios of the reinforced composite construct as a function of incubation time in PBS, ranging from ca. 25% (w/w) for the PCL/G25 to ca. 50% (w/w) for PCL/G10 compared to ca. 30% (w/w) for the G25 hydrogel without reinforcement after incubation for 48 h at 37° C. (FIG. 5A). HC swelled up to 82% (w/w) in similar settings.

Collagenase, matrix metalloproteinase (MMP), and other proteolytic enzymes have high concentrations and activity in the injured area to help tissue regeneration and modeling. These enzymes hydrolyze proteins and weaken the protein-based construct unless there is compensatory tissue regeneration at a higher rate. Thus, the stability of an implant against enzymatic degradation is crucial. FIG. 5B illustrates the retention of reinforced composites constructs in a highly concentrated collagenase solution and their enzymatic stability against degradation. The enzymatic degradation rate of specimens is as follows: hydrogel without nanofiber reinforcement>HC>reinforced constructs (with the degradation rate depending on hydrogel of the composite). For instance, the hydrogel without nanofiber reinforcement almost fully dissolved in 24 h; HC degraded in 36 h; however, reinforced constructs exhibited more than 40% retention after 36 h of treatment. G-GMA/N-vinylpyrrolidone copolymeric hydrogel constructs with [VP]=5-10% showed 20% retention after 36 h. Visible light crosslinked GelMA was shown to degrade in less than 4 h in a similar setting. Aligned and random plant-derived recombinant human collagen was shown to completely degrade by 8 h and 16 h, respectively, in similar conditions. Recombinant human collagen-based hydrogels were also degraded by 12 h in similar conditions. The higher stability of the electrospun reinforced hydrogels could be attributed to the restriction of the accessibility of the enzyme to the cleavage sites of the hydrogel by the PCL nanofibers, along with the entrapment and entanglement of the degraded units within those PCL nanofibrous networks.

Cells are dynamically involved in sustaining the structural integrity and function of the tissue. Most of the energy for such maintenance originates from glucose catabolism. Due to its avascular nature, the cornea depends on the diffusion of nutrients from aqueous humor to the epithelium and stromal cells. If the diffusion of nutrition is interrupted, neither the limbus nor the tears can provide enough nutrients to preserve corneal function, which may lead to corneal melt and Necrosis. Glucose diffusion studies demonstrated that reinforcement of the hydrogel with PCL nanofibers decreased the permeability of the construct from ca. 2.92·10⁻⁶ cm⁻²/s for the nonreinforced matrix of hydrogel to ca. 2.82·10⁻⁶ cm⁻²/s in [hydrogel]-dependent manner (FIG. 5C). However, all constructs showed higher permeability than HC, suggesting their suitability for transplantation to allow nutrition and signaling factors to diffuse in and reach the cells while enabling the metabolic by-products to diffuse out.

### Biocompatibility

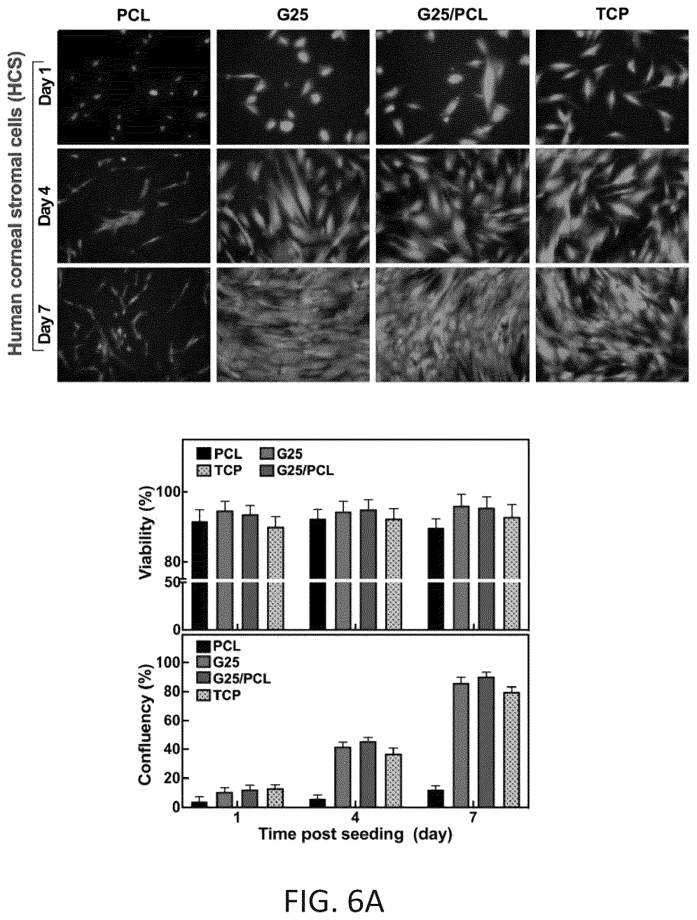

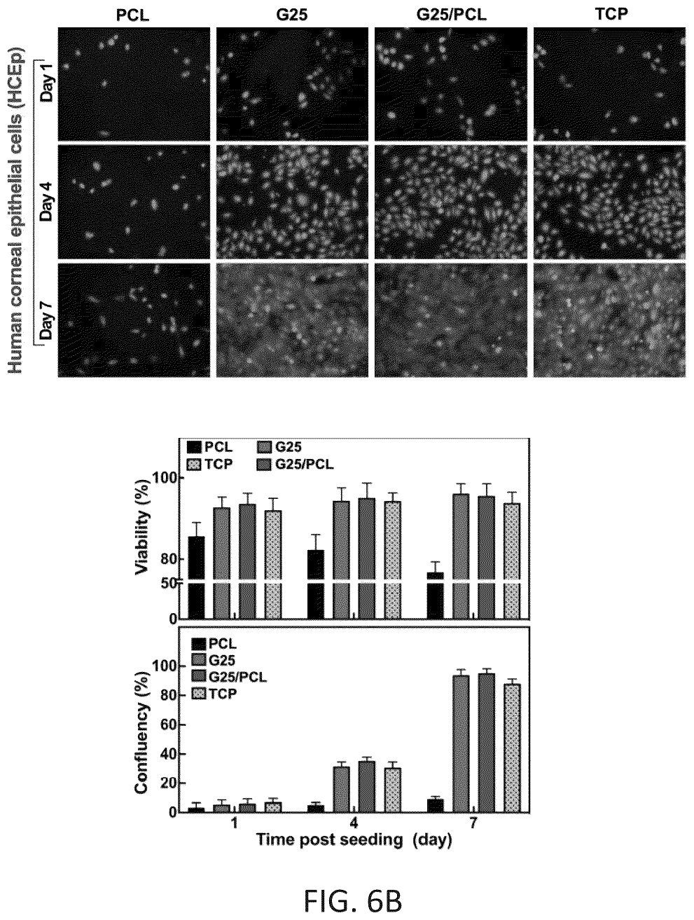

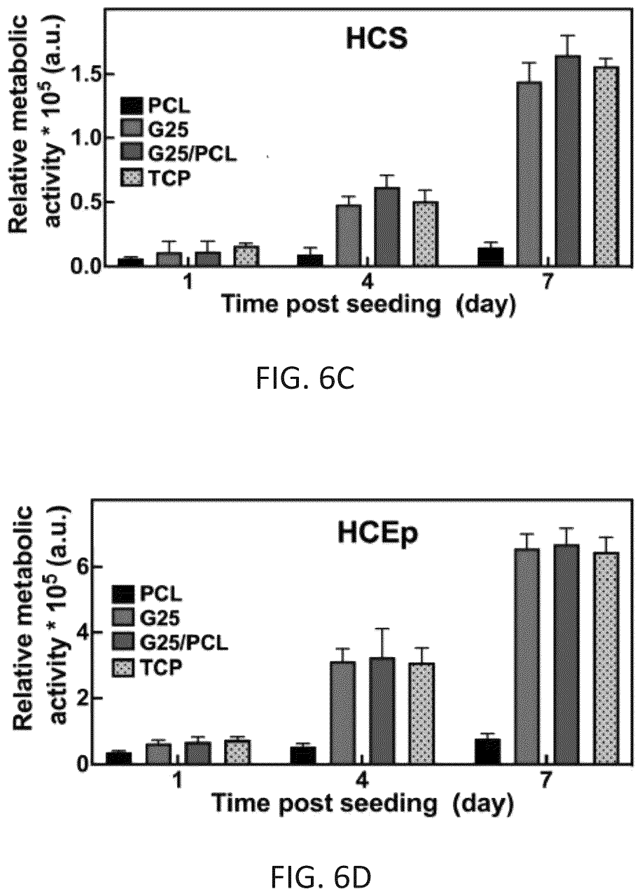

Efficacious biointegration between an artificial tissue (e.g., cornea) and the host tissue relay on the function of cells (e.g., corneal stromal cells (CS)), which under optimum conditions migrate from the host tissue into the artificial construct. Thus, CS should have favorable interactions with the construct to adhere, proliferate, and generate ECM to regenerate healthy tissue. Corneal epithelial cells should also favorably interact with the construct to generate stratified corneal epithelium. To evaluate the biological interactions of corneal cells with the reinforced constructs, we performed an in-vitro cell biocompatibility test using human CS (HCS) and human corneal epithelial cells (HCEp) cell lines (FIGS. 6A-6D). The Live-Dead assay revealed that HCS cultured on hydrogel with and without enforcement have similar cellular viabilities (ca. 94-97%) at all time points (1, 4, and 7 days of post culture) and have slightly higher viability than those cultured on reference TCP (ca. 90-92%) or PCL mats (ca. 89-92%) (FIG. 6A). Additionally, HCS cultured on hydrogel with and without reinforcement demonstrated similar spreading patterns and morphology to those on TCP controls yet slightly higher cell confluency (FIG. 6A) at all time points. However, HCS cells on the PCL tended to have elongated shapes, spread very poorly, and exhibited much lower confluency than other groups due to a lack of cell adhesion on the PCL structure/surface and its hydrophobic nature. HCEp showed almost a similar trend, with the highest viabilities for cells cultured on constructs with and without reinforcement (ca. 92-96%), compared to those on reference TCP (ca. 92-94%) and PCL (ca. 76-85%) as shown in FIGS. 6B. Likewise, the cells on the constructs demonstrated similar spreading patterns and morphologies to those of TCP yet showed slightly higher confluency. However, HCEp cells on the PCL tended to have a round shape, spread poorly, and showed much lower confluency (ca. 9% after 7 days of cell culture) than other groups (ca. 87-95%). The cells' viability, confluency, spreading patterns, and morphology were similar for constructs with and without PCL reinforcement, suggesting that the integration of PCL nanofibers did not negatively impact the biocompatibility of the constructs.

AlamarBlue testing showed that both HCS and HCEp cells seeded on the constructs demonstrate gradual increases in relative fluorescence intensities as a function of incubation time—in a comparatively similar trend to those on reference TCP—indicating an increase in metabolic activity of cultured cells (FIGS. 6C-6D). However, cells cultured on PCL nanofibers showed much lower metabolic activity over time, demonstrating that the PCL mats are not a favorable surface for cells to attach, spread, and proliferate, as also witnessed by the live-dead assay.

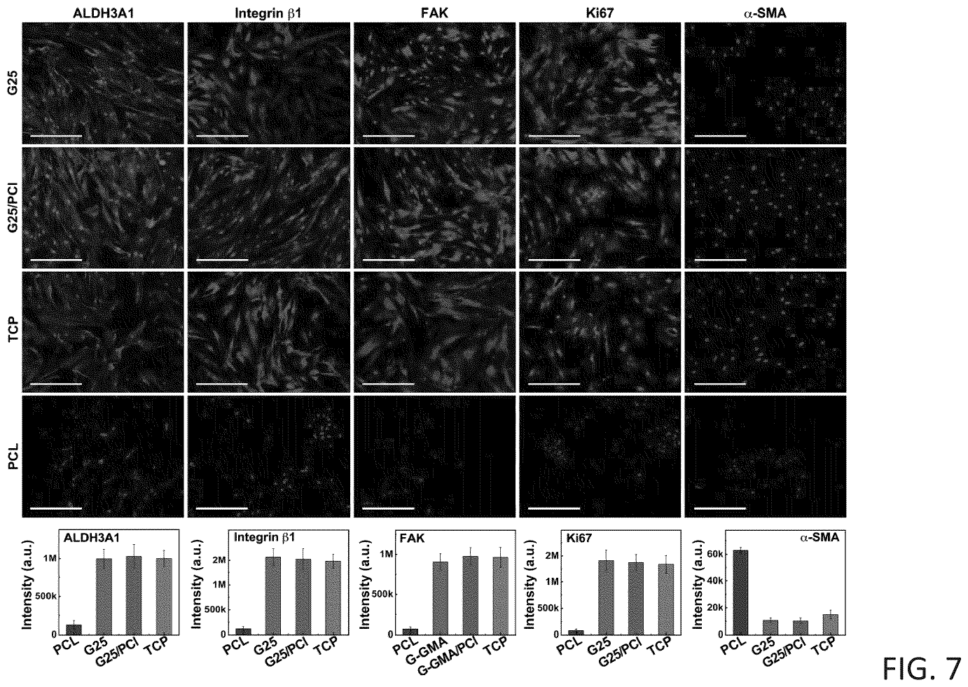

To further comprehend the interaction of human corneal cells with the reinforced composite, we studied the expression of specific markers, including adhesion, proliferation, and proinflammatory markers, using ICC (FIG. 7). Cultured HCS cells expressed ALDH3A1 (keratocytespecific marker) on both hydrogel and reinforced hydrogel at a similar level to those on TCP, suggesting that the construct does not impact the physiologically normal keratocyte phenotype. However, HCS cells cultured on PCL showed limited expression of ALDH3A1. HCS cells express several collagen-binding integrins, including α2β1, α3β1, α5β1, and α6β, which regulate cell interactions with the extracellular matrix. Integrins also serve as bidirectional conduits for signals to regulate mechanical stress. HCS cells cultured on both constructs expressed a high level of integrin β1, which was similar to that on TCP, indicating that the construct is a favorite substrate for adhesion of the stromal cells. However, the expression of integrin β1 was minimal in the cells cultured on PCL. We also found a high level of focal adhesion kinase (FAK) expression, associated with cellular adhesion and spreading, on the cells cultured on both constructs and TCP. Yet, FAK expression appeared relatively lower on PCL. Additionally, most of the HCS cells cultured on both constructs expressed a high level of proliferation marker Ki67, similar to that on TCP. Yet, only a limited number of cells cultured on

PCL expressed any Ki67 as expected. Notably, there was almost no expression of alpha-smooth muscle actin (α-SMA), which is associated with pro-inflammatory and fibrotic responses, on both constructs and TCP, indicating the constructs did not induce a phenotypic change of HCS cells into myofibroblasts. These data suggest that the reinforced construct is biocompatible and promotes cell adhesion and proliferation.

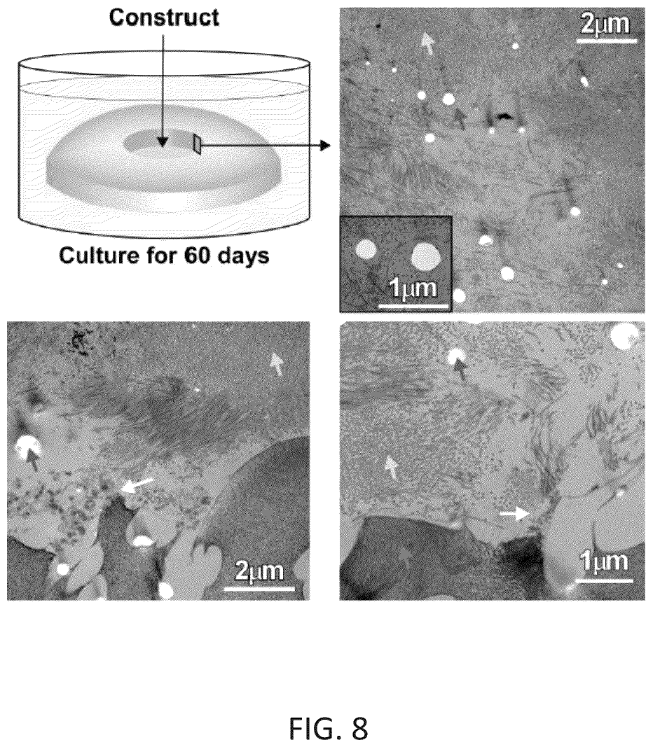

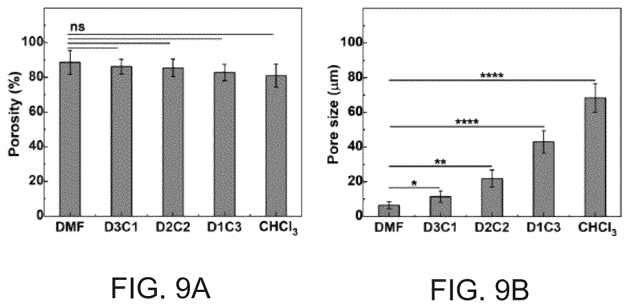

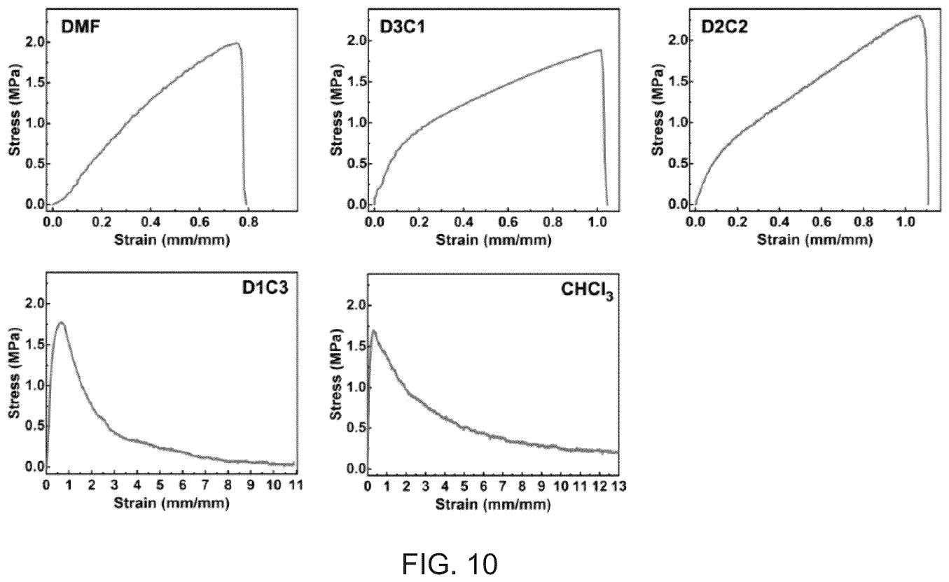

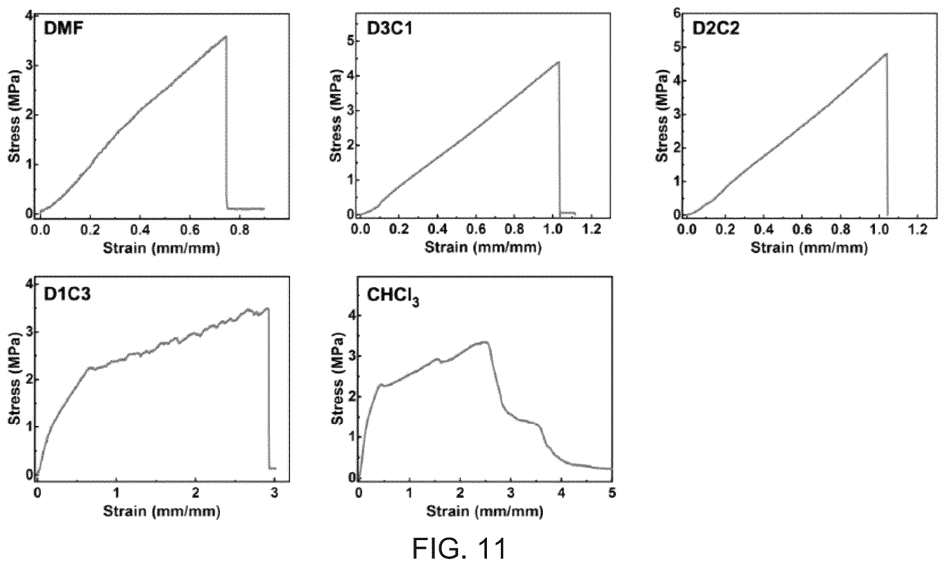

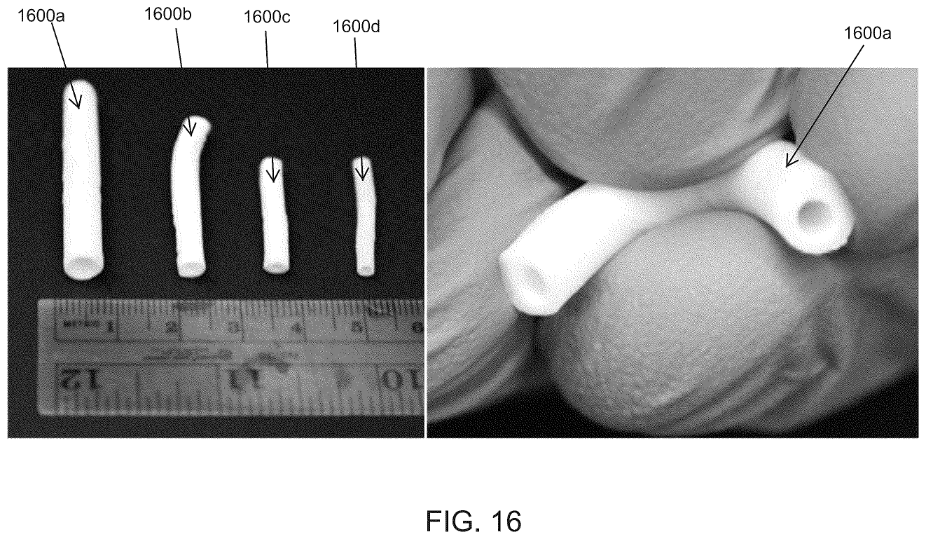

To study the migration of HCS into a composite reinforced construct and its biointegration with the HC, we performed an ex vivo study in which we implanted the construct in the center of the donor cornea, as shown in FIG. 8, and cultured it for 2 months. Transmission electron microscopy (TEM) analysis from the interface of the construct and host tissue showed that the construct (indicated by pink arrows) is degrading (shown by white arrows) by the stromal cells as the new tissue regenerates. Since the degradation of the hydrogel matrix is much faster than PCL nanofibers, those fibers (shown by red arrows) stay behind, which then be surrounded by the newly formed collagen. It is important to note that the newly generated collagen at the interface is first non-organized (shown by green arrows), then becomes organized (yellow arrows). It is essential to mention that our developed reinforced composite construct will function as the skirt to be sutured into the host tissue, while the central part (G25) serves as optical windows to transmit the light into the lens and then the retina. Therefore, the engineered construct can be used in deep anterior lamellar keratoplasty (DALK) and penetrating keratoplasty, in which both cases, the skirt will be reinforced construct, and the core will be G-GMA hydrogel. In addition, the reinforced construct can be used as a donor tissue substitute in keratoprosthesis (KPro) implantation, which is indicated for patients with corneal blindness not amenable to standard corneal transplantation. Besides corneal transplantation, our reinforced system can be used to generate other suturable and transplantable tissue constructs with desired mechanical properties, such as tendons, ligaments, skin, heart valves, and blood vessels. For example, FIG. 16 shows hydrogel-nanofiber artificial blood vessels 1600a, 1600b, 1600c, 1600d according to aspects of the present disclosure. In addition, our reinforced system can be combined with 3D-bioprinting and other fabrication techniques to generate suturable cell-laden constructs with precise configuration and functionality required by the specific biomedical field.

By integrating hydrogels with high porosity electrospun PCL mats, layer-by-layer stacking, and crosslinking the system, the suturablity and mechanical, structural, and chemical properties of a soft hydrogel are significantly and synergistically improved, approaching or even surpassing those of native corneal tissue. The synergistic effect could be modulated by altering the PCL nanofibers' size and hydrogel concentration in the matrix to generate reinforced constructs with varying mechanical, structural, chemical properties, and biological properties for biomedical application and, in particular, tissue transplantation.

While the invention has been described with reference to preferred embodiments, those skilled in the art will appreciate that certain substitutions, alterations and/or omissions may be made to the embodiments without departing from the spirit of the invention. Accordingly, the foregoing description is meant to be exemplary only, and should not limit the scope of the invention.

1. A method of making a medical implant, the method comprising:
(a) electrospinning a polymer solution to form a polymer fiber mat;
(b) diffusing a solution including a crosslinkable hydrogel into the polymer fiber mat to form a hydrogel-infused mat; and
(c) irradiating the hydrogel-infused mat to crosslink the hydrogel and form the medical implant.

2. The method of claim 1 wherein:
step (a) comprises electrospinning the polymer solution to form a plurality of polymer mats.

3. The method of claim 2, wherein:
step (b) comprises diffusing a solution including crosslinkable hydrogel into the plurality of polymer fiber mats to form a plurality of hydrogel-infused mats; and
further stacking the plurality of polymer fiber mats to form a stack of hydrogel-infused mats.

4. The method of claim 3, wherein:
step (c) comprises irradiating the stack of hydrogel-infused mats to crosslink the hydrogel and form the medical implant.

5. The method of claim 1 wherein:
step (c) comprises molding the hydrogel-infused mat and irradiating the hydrogel-infused mat to crosslink the hydrogel and form the medical implant.

6. The method of claim 1 wherein:
step (c) comprises irradiating the hydrogel-infused mat to crosslink the hydrogel to form a construct, forming an opening in the construct, filling the opening with an additional crosslinkable hydrogel to form a filled construct, and irradiating the additional crosslinkable hydrogel to form a corneal implant.

7. The method of claim 1 wherein:
the solution includes poly(e-caprolactone) (PCL).

8. The method of claim 1 wherein:
the solution includes one of a polypeptide biopolymer or a polysaccharide biopolymer.

9. The method of claim 1 wherein:
the solution includes one of gelatin and its derivates.

10. The method of claim 1 wherein:
the solution includes gelatin glycidyl methacrylate (G-GMA).

11. The method of claim 2 wherein:
each polymer mat includes fibers of varying diameters, varying orientations of the fibers, or both.

12. The method of claim 1 wherein:
step (c) comprises irradiating the hydrogel-infused mat using a visible light source.

13. The method of claim 1 wherein:
step (c) comprises irradiating the hydrogel-infused mat using a light emitting diode (LED).

14. The method of claim 1 further comprising:
step (b) comprises shaving off an excess of the solution from the hydrogel-infused mat diffusing the solution into the polymer fiber mat.

15. A medical implant comprising:
a polymer fiber stack comprising electrospun polymeric fibers; and
a crosslinked hydrogel matrix,
wherein the polymer fiber stack is embedded within the crosslinked hydrogel matrix.

16. The medical implant of claim 15 wherein:
the polymer fiber stack comprises a stack of a plurality of polymer mats, each polymer mat comprising the electrospun polymeric fibers.

17. The medical implant of claim 15 wherein:
the electrospun polymeric fibers comprise electrospun poly(e-caprolactone) (PCL) polymer fibers.

18. The medical implant of claim 15 wherein:
the electrospun polymeric fibers include fibers of varying diameters, varying orientations, or both.

19. The medical implant of claim 15 wherein:
the hydrogel matrix includes one of a polypeptide biopolymer or a polysaccharide biopolymer.

20. The medical implant of claim 15 wherein:
the hydrogel matrix includes one of gelatin and its derivates.

21. The medical implant of claim 15 wherein:
the hydrogel matrix includes gelatin glycidyl methacrylate (G-GMA).

22. The medical implant of claim 15 wherein:
the polymer fiber stack includes an opening, and
an additional crosslinked hydrogel matrix positioned in the opening.

23. The medical implant of claim 22 wherein:
the additional crosslinked hydrogel matrix includes one of gelatin and its derivates.

24. The medical implant of claim 22 wherein:
the additional crosslinked hydrogel matrix includes gelatin glycidyl methacrylate (G-GMA).

25. The medical implant of claim 22 wherein:
the additional crosslinked hydrogel matrix is transparent.

26. The medical implant of claim 22 wherein:
the crosslinked hydrogel matrix, the opening, and the additional crosslinked hydrogel matrix are each dimensioned such that the implant is a corneal implant.

27. The medical implant of claim 16 wherein:
each polymer mat has an ultimate tensile strength in a range of 2.5 to 5.5 MPa.

28. The medical implant of claim 16 wherein:
each polymer mat has a tensile modulus in a range of 2.5 to 5.5 MPa.

29. The medical implant of claim 16 wherein:
each polymer mat has a compressive modulus in a range of 2 to 4 MPa

30. The medical implant of claim 16 wherein:
each polymer mat has a contact angle in a range of 130° to 135°.

31. The medical implant of claim 16 wherein:
each polymer mat has a BSA permeability in a range of 5 to 20 cm²/s.

32. The medical implant of claim 16 wherein:
an adhesion strength between each polymer fiber mat embedded in crosslinked hydrogel matrix is in a range of 0.2 to 0.7 MPa.

33. The medical implant of claim 15 wherein:
the implant has a burst pressure in a range of 75 to 275 kPa.

34. The medical implant of claim 15 wherein:
the implant has a suture rupture force in a range of 3 to 6N.

35. The medical implant of claim 15 wherein:
the implant has a glucose diffusion in a range of 2 to 4 cm²/s.
